(19) United States
(12) Patent Application Publication
(10) Pub. No.: US 2026/0123826 A1
(43) Pub. Date: May 7, 2026

(54) ENDOSCOPE DEVICE AND STEERING MODULE THEREOF

(71) Applicant: INDUSTRIAL TECHNOLOGY RESEARCH INSTITUTE, Hsinchu (TW)

(72) Inventors: Hao-Yan WU, Taoyuan City (TW); Yan-Ting CHEN, Linluo Township (TW); Chien-Yu WU, Kouhu Township (TW); Su-Jhen LIN, Kaohsiung City (TW)

(73) Assignee: INDUSTRIAL TECHNOLOGY RESEARCH INSTITUTE, Hsinchu (TW)

(21) Appl. No.: 19/002,448

(22) Filed: Dec. 26, 2024

(30) Foreign Application Priority Data
Nov. 5, 2024 (TW) 113142341

(51) Int. Cl. A61B 1/005

(52) U.S. Cl. CPC A61B 1/0057

(57) ABSTRACT

A steering module includes a module outer rotating element and a module inner rotating component. The module outer rotating element includes a first module matching portion. The module inner rotating element is rotatable relative to the module outer rotating element and passes through the module outer rotating component and includes a second module matching portion. The first module matching portion and the second module matching portion are configured to connect with a base of an endoscope device.

This application claims the benefit of Taiwan application Serial No. 113142341, filed November 5, 2024, the disclosure of which is incorporated by reference herein in its entirety.

### TECHNICAL FIELD

The disclosure relates in general to an endoscope device and a steering module thereof.

### BACKGROUND

An endoscope device have been widely used in medical, industrial and other fields, which may enter the interior of an to-be-tested object, observe an internal tissues of the to-be-tested object, and even properly process the internal tissues. However, the endoscope device needs to be thoroughly disinfected after each use, and thus it is quite time-consuming. Therefore, how to improve the aforementioned conventional problems is one of the goals of those in this technical field.

### SUMMARY

According to an embodiment, a steering module of an endoscope device is provided. The steering module includes a module outer rotating component and a module inner rotating component. The module outer rotating component includes a first module matching portion. The module inner rotating component passes through the module outer rotating component and is rotatable relative to the module outer rotating component and includes a second module matching portion. The first module matching portion and the second module matching portion are configured to connect with a base of the endoscope device.

According to another embodiment, an endoscope device is provided. The endoscope device includes a steering module and a base. The steering module includes a module outer rotating component and a module inner rotating component. The module outer rotating component includes a first module matching portion. The module inner rotating component passes through the module outer rotating component and is rotatable relative to the module outer rotating component and includes a second module matching portion. The base includes a base outer rotating component and a base inner rotating component. The base outer rotating component has a through hole and a first base matching portion. The base inner rotating component is disposed in the through hole, is rotatable relative to the base outer rotating component and has a second base matching portion. The first base matching portion and the second base matching portion are configured to connect to the first module matching portion and the second module matching portion of the steering module respectively.

The above and other aspects of the disclosure will become better understood with regard to the following detailed description of the preferred but non-limiting embodiment (s). The following description is made with reference to the accompanying drawings.

### BRIEF DESCRIPTION OF THE DRAWINGS

FIG. 1 illustrates a schematic diagram of an endoscope device according to an embodiment of the present disclosure;

FIGS. 2A and 2B illustrate schematic diagrams of exploded views of the endoscope device in FIG. 1 along different viewing angles;

FIG. 3 illustrates a schematic diagram of an exploded view of the base in FIG. 1;

FIG. 4 illustrates a schematic diagram of a cross-sectional view of the base in FIG. 2A along XZ plane;

FIGS. 5A and 5B illustrate schematic diagrams of exploded views of the first steering module in FIG. 1 along different viewing angles;

FIG. 6 illustrates a schematic diagram of a cross-sectional view of the endoscope device in FIG. 1 along a direction 6-6';

FIG. 7 illustrates a schematic diagram of a bottom view of the endoscope device in FIG. 1;

FIG. 8 illustrates a schematic diagram of a cross-sectional view of the endoscope device in FIG. 1 along a direction 8-8';

FIG. 9 illustrates a partial schematic diagram of the interior of the first steering module in FIG. 1;

FIG. 10 illustrates a schematic diagram of a bottom view of the base 100 and the second steering module in FIG. 1 after assembly;

FIG. 11 illustrates a schematic diagram of a cross-sectional view of the endoscope device along a direction 11-11';

FIGS. 12A and 12B illustrate schematic diagrams of an exploded view of the second steering module in FIG. 10 viewed from different viewing angles; and

FIG. 13 illustrates a schematic diagram of an exploded view of the second steering module in FIG. 12A.

### DETAILED DESCRIPTION

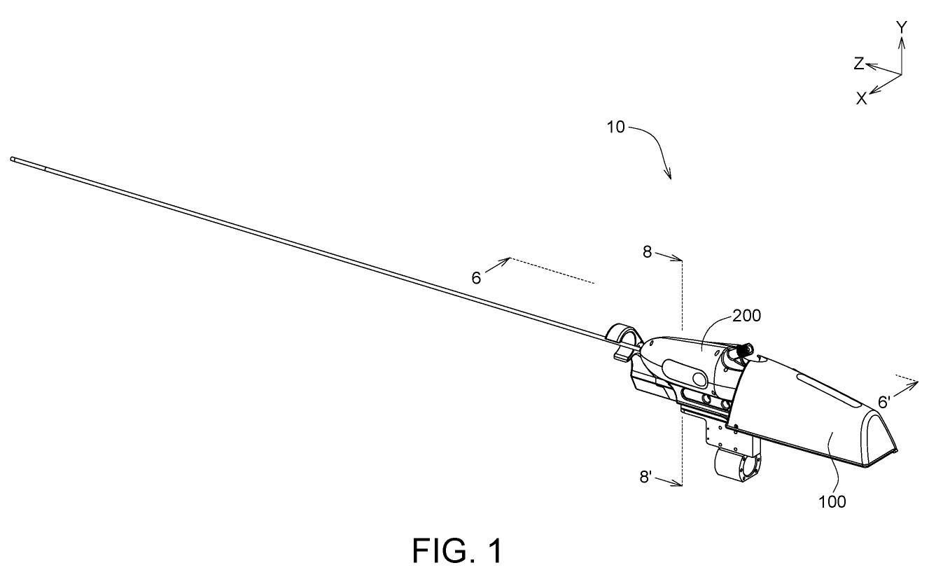

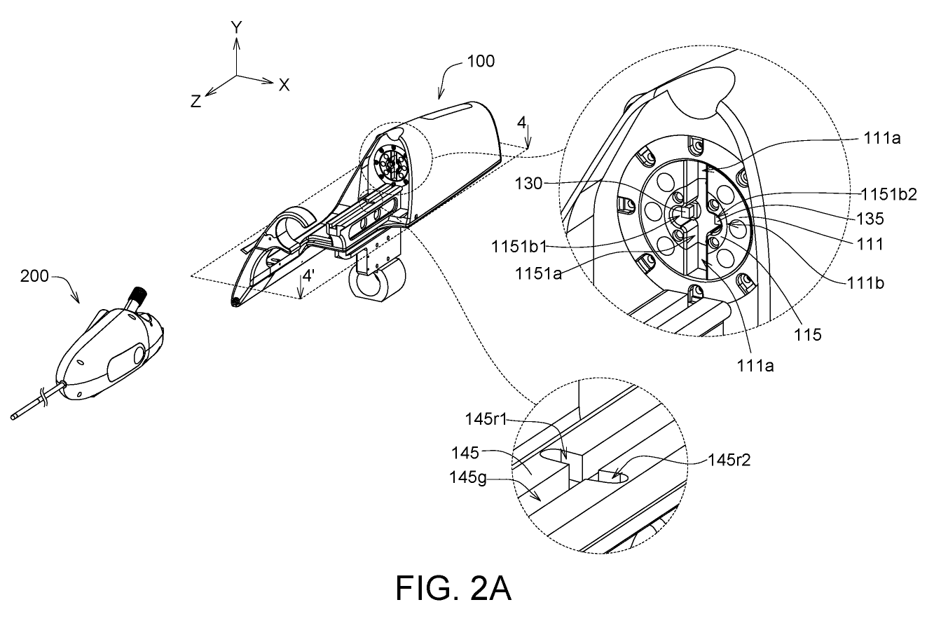

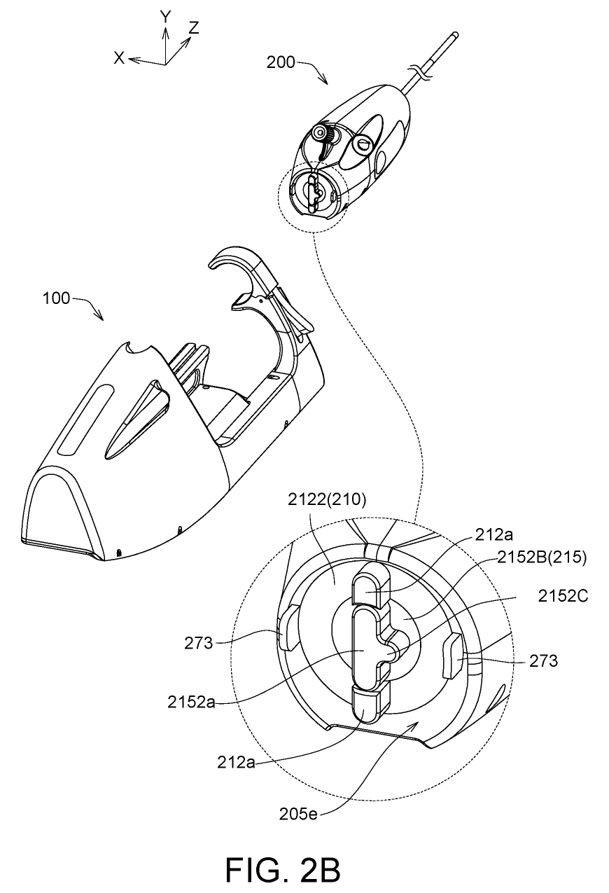

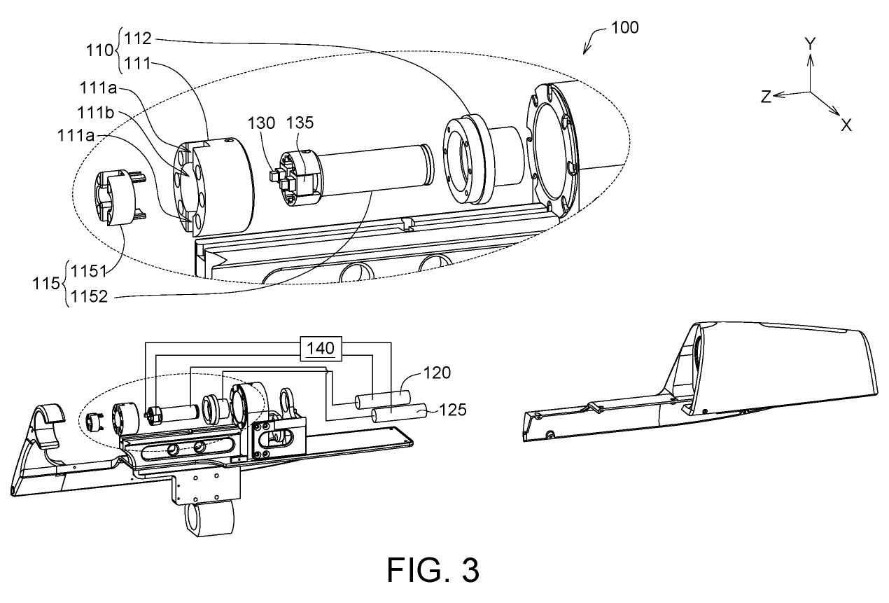

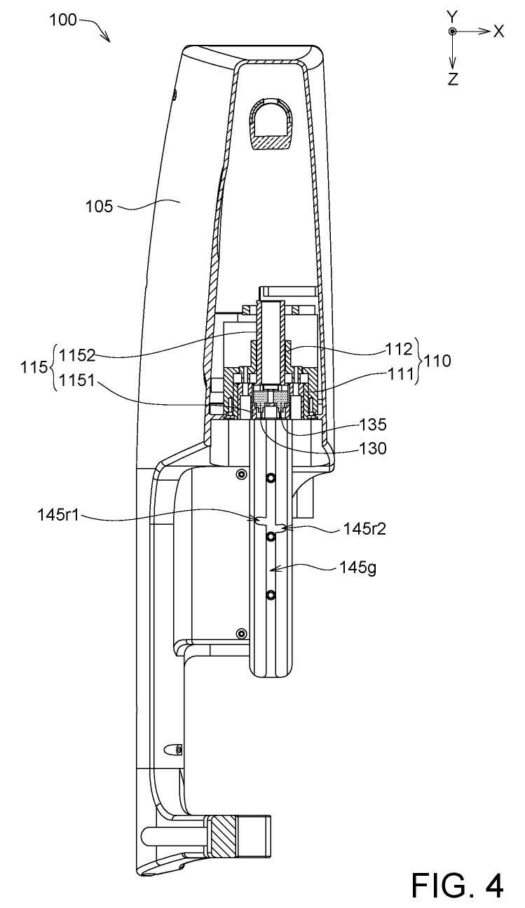

Referring to FIGS. 1, 2A, 2B, 3 and 4, FIG. 1 illustrates a schematic diagram of an endoscope device 10 according to an embodiment of the present disclosure, FIGS. 2A and 2B illustrate schematic diagrams of exploded views of the endoscope device 10 in FIG. 1 along different viewing angles, FIG. 3 illustrates a schematic diagram of an exploded view of the base 100 in FIG. 1, and FIG. 4 illustrates a schematic diagram of a cross-sectional view of the base 100 in FIG. 2A along XZ plane.

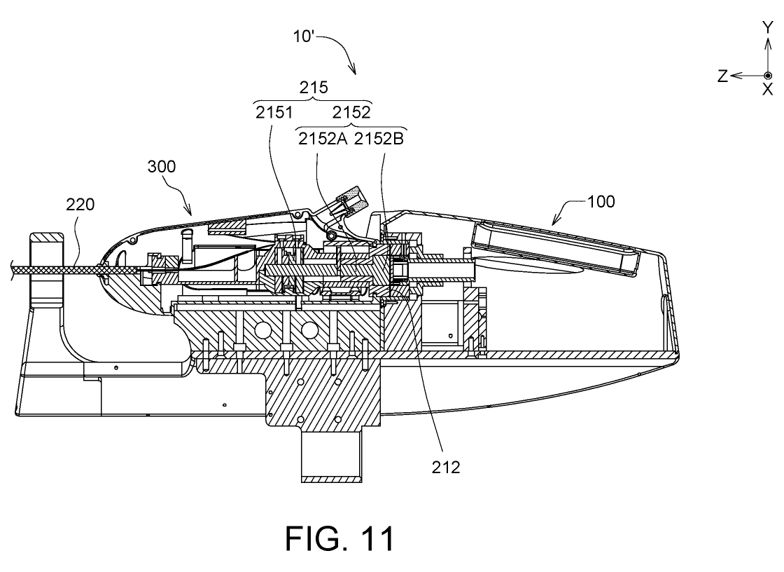

The endoscope device 10 includes the base 100 and a first steering module 200. In the present embodiment, the second steering module 300 (as illustrated in FIGS. 11, 12A to 12B and 13) may also be connected to the base 100. In other words, the base 100 of the embodiment of the present disclosure may be connected to one of a variety of steering modules, that is, a variety of steering modules may share the same base 100. In addition, although not illustrated, the base 100 may be connected to a robot arm which may operate the endoscope device 10.

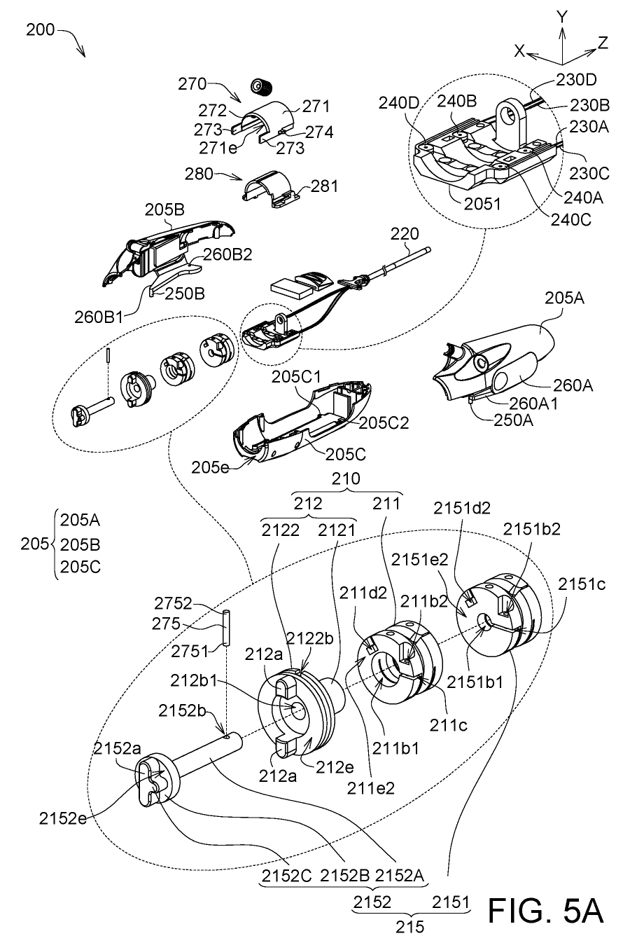

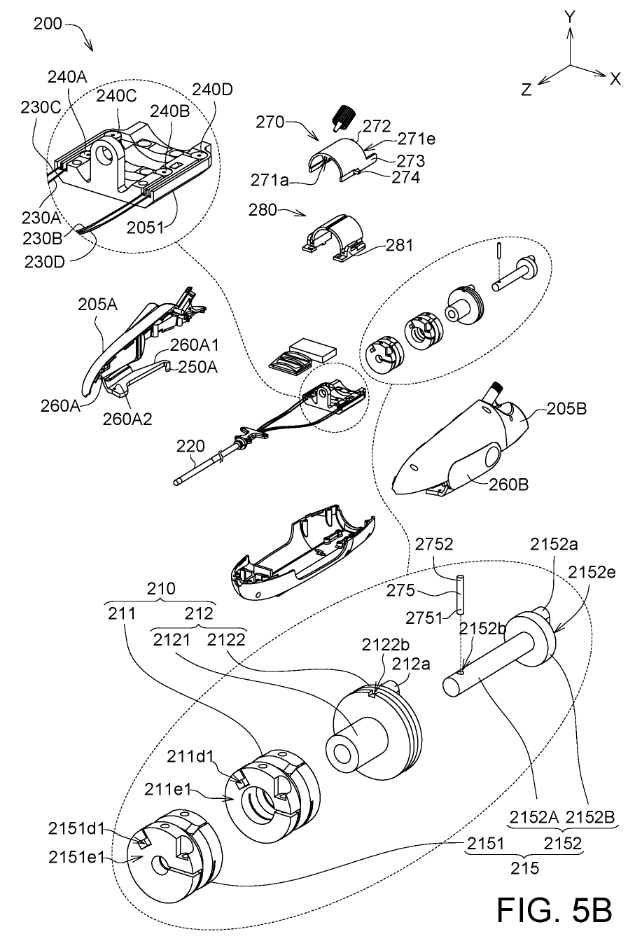

The endoscope device 10 may be applied in the medical field or industrial engineering field. In the medical field, the endoscope device 10 may be applied to a robot arm. The endoscope device 10 may control the movement of a flexible tube 220 (the flexible tube 220 is illustrated in FIG. 5A), such as forward, backward, rotation and/or turning. The flexible tube 220 includes a tool, such as a needle, a cauterizer, a micro camera, a scraper (used to scrape off foreign matter, etc.). The flexible tube 220 may enter the interior of the to-be-tested object to process the internal structure of the to-be-tested object. The to-be-tested object may be a living body (for example, a human or an animal, or a natural hole or passage such as a bronchi or intestine in a living body) or a non-living body (for example, an object other than living things, such as a building, a processing machine, ground, electronic device, liquid channel, gas channel, etc.). In the case of living organisms, the internal structure includes abnormal tissues, such as tumor and polyp. Although not illustrated, the endoscope device 10 further includes a camera, which may be disposed at a front end of the flexible tube 220 to capture a front image, wherein the image may be transmitted to the controller through wire or wireless communication technology (described later).

The following introduces the structure of the base.

As illustrated in FIGS. 2A, 2B, 3 and 4, the base 100 includes a base shell 105, a base outer rotating component 110, a base inner rotating component 115, a first driver 120, a second driver 125, a first micro switch 130, a second micro switch 135, a controller 140 and a slide rail 145.

As illustrated in FIGS. 2A and 3, the base outer rotating component 110 has at least a first base matching portion 111a and a through hole 111b. The base inner rotating component 115 is disposed in the through hole 111b, is rotatable relative to the base outer rotating component 110 and has a second base matching portion 1151a. The first base matching portion 111a and the second base matching portion 1151a are configured to connect with one of the first steering module 200 and the second steering module 300. As a result, a variety of the steering modules may share the same base 100. In addition, the steering module may be a disposable module (for reducing the risk of the secondary infection), so that the endoscope device does not require the disinfection procedure after each use, and the base may continue to be used.

The base outer rotating component 110 includes at least one base outer rotating component 111 and a base outer rotating shaft 112, wherein the base outer rotating component 111 has the aforementioned first base matching portion 111a and the through hole 111b. The base outer rotating shaft 112 may be connected to the base outer rotating component 111. For example, the base outer rotating shaft 112 and the base outer rotating component 111 may be fixed to each other through at least one screw (not illustrated). As a result, when the base outer rotating shaft 112 rotates, the base outer rotating component 111 may be driven to move together. In the present embodiment, the base outer rotating shaft 112 and the base outer rotating component 111 may rotate around +/-Z axis. In the present embodiment, the first base matching portion 111a is, for example, a groove. In addition, the number of the first base matching portions 111a is, for example, two, which are respectively located on two opposite sides of the through hole 111b.

The base inner rotating component 115 includes a base inner rotating portion 1151 and a base inner rotating shaft 1152, wherein the base inner rotating portion 1151 has the aforementioned second base matching portion 1151a (the second base matching portion 1151a is illustrated in FIG. 2A). The base inner rotating shaft 1152 may be connected to the base inner rotating portion 1151. For example, the base inner rotating shaft 1152 and the base inner rotating portion 1151 may be fixed to each other through at least one screw (not illustrated). As a result, when the base inner rotating shaft 1152 rotates, the base inner rotating portion 1151 may be driven to move together. In the present embodiment, the base inner rotating shaft 1152 and the base inner rotating portion 1151 may rotate around Z-axis. In the present embodiment, the second base matching portion 1151a is, for example, a groove.

In the present embodiment, the base outer rotating component 110 and the base inner rotating component 115 may be coaxially disposed. As a result, the base outer rotating component 110 and the base inner rotating component 115 may rotate coaxially.

In an embodiment, when the base 100 is in a standby state (for example, the base outer rotating component 111 and the base inner rotating component 115 of the base 100 do not rotate), the second base matching portion 1151a and the first base matching portion 111a may be arranged in a straight line.

As illustrated in FIGS. 2A and 3, one of the base outer rotating component 110 and the base inner rotating component 115 may have a first positioning portion and a second positioning portion. The first positioning portion is allowed to connect with one of the first steering module 200 and the second steering module 300, and the second positioning portion is allowed to connect with the other one of the first steering module 200 and the second steering module 300. For example, the base inner rotating portion 1151 of the base inner rotating component 115 has a first positioning portion 1151b1 and a second positioning portion 1151b2. The first positioning portion 1151b1 and the second positioning portion 1151b2 are respectively disposed on two opposite sides of the second base matching portion 1151a. The first positioning portion 1151b1 is allowed to connect with the first steering module 200, and the second positioning portion 1151b2 is allowed to connect with the second steering module 300. In the present embodiment, the first positioning portion 1151b1 is, for example, a through hole, which may expose the first micro switch 130 and/or the second positioning portion 1151b2 is, for example, a through hole, which may expose the second micro switch 135.

As illustrated in FIG. 3, the first driver 120 is connected to the base outer rotating component 110 to drive the base outer rotating component 110 to rotate. Furthermore, the first driver 120 may be connected to the base outer rotating shaft 112 of the base outer rotating component 110. The second driver 125 is connected to the base inner rotating component 115 to drive the base inner rotating component 115 to rotate. Specifically, the second driver 125 may be connected to the base inner rotating shaft 1152. In an embodiment, the first driver 120 and/or the second driver 125 are, for example, motors.

As illustrated in FIG. 3, the first micro switch 130 and the second micro switch 135 may be disposed on the base inner rotating shaft 1152. The first micro switch 130 is configured to detect one of the first steering module 200 and the second steering module 300, and the second micro switch 135 is configured to detect the other one of the first steering module 200 and the second steering module 300. For example, the position of the first micro switch 130 corresponds to the first positioning portion 1151b1, while the position of the second micro switch 135 corresponds to the second positioning portion 1151b2. As a result, when the first steering module 200 is connected to the base 100, the first micro switch 130 may be triggered, and accordingly the first micro switch 130 transmits a first trigger signal. Similarly, when the second steering module 300 is connected to the base 100, the second micro switch 135 may be triggered, and accordingly the second micro switch 135 transmits a second trigger signal.

As illustrated in FIG. 3, the controller 140 is electrically connected to the first driver 120, the second driver 125, the first micro switch 130 and the second micro switch 135. The controller 140 may control the operation of the first driver 120 to drive the base outer rotating component 110 to rotate. The controller 140 may control the operation of the second driver 125 to drive the base rotating component 115 to rotate. When the controller 140 receives the first trigger signal from the first micro switch 130, the endoscope device 10 may enter (or activate) a four-way steering mode. When the controller 140 receives the second trigger signal from the second micro switch 135, the endoscope device 10 may enter (or activate) a two-way steering mode.

As illustrated in FIGS. 2A, 3 and 4, the slide rail 145 is disposed on the base shell 105 and has a guide groove 145g, a first fastening groove 145r1 and a second fastening groove 145r2. Both the first steering module 200 and the second steering module 300 may slide in the guide groove 145g and be guided by the guide groove 145g to be locked in the first fastening groove 145r1 and the second fastening groove 145r2. The first fastening groove 145r1 may be connected with any one of the first steering module 200 and the second steering module 300, and the second fastening groove 145r2 may also be connected with any one of the first steering module 200 and the second steering module 300. In other words, both the first steering module 200 and the second steering module 300 may be connected to the first fastening groove 145r1 and the second fastening groove 145r2. The first fastening groove 145r1 and the second fastening groove 145r2 may extend to the guide groove 145g. In addition, the first fastening groove 145r1 and the second fastening groove 145r2 may be staggered by a distance along an extension direction of the guide groove 145g (for example, along Z-axis direction). In other words, the first fastening groove 145r1 and the second fastening groove 145r are not arranged in a straight line along the X-axis.

The structure of the first steering module is described below.

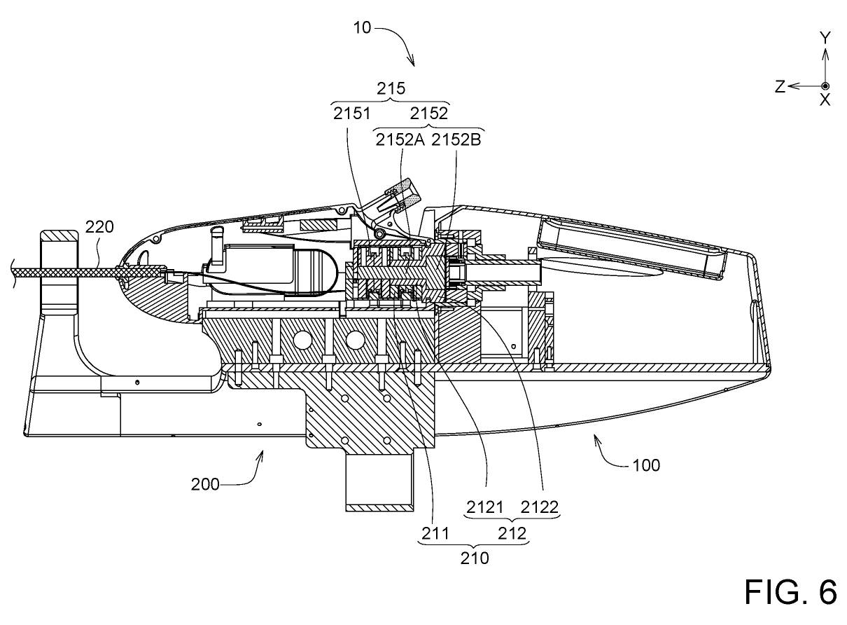

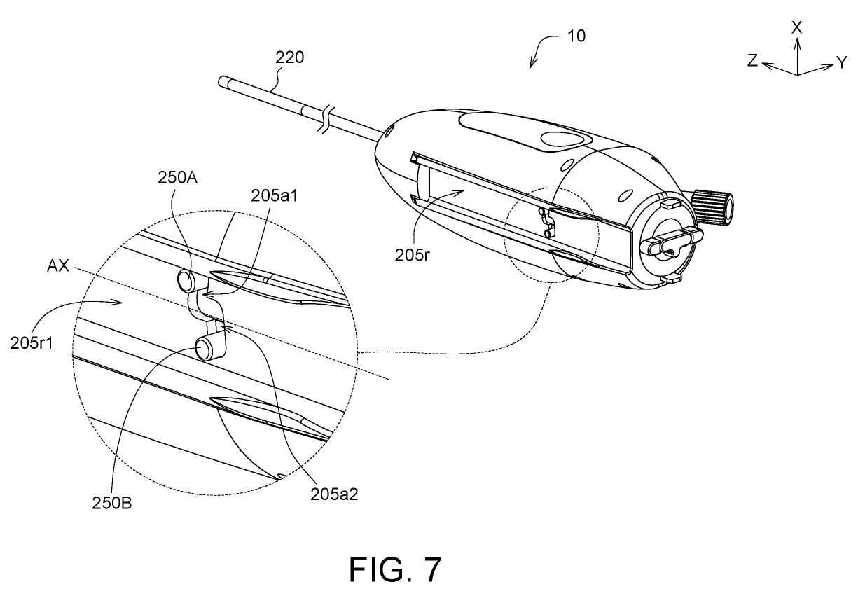

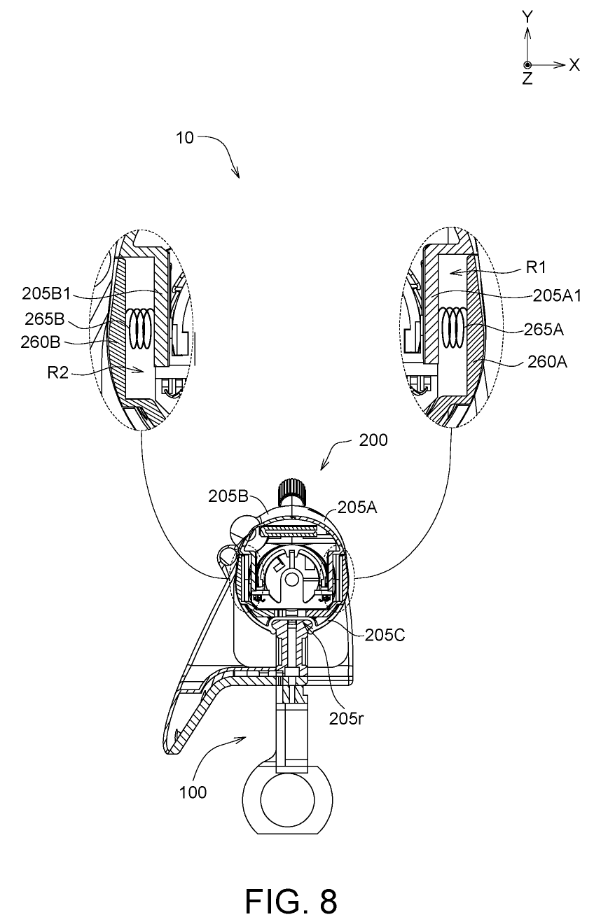

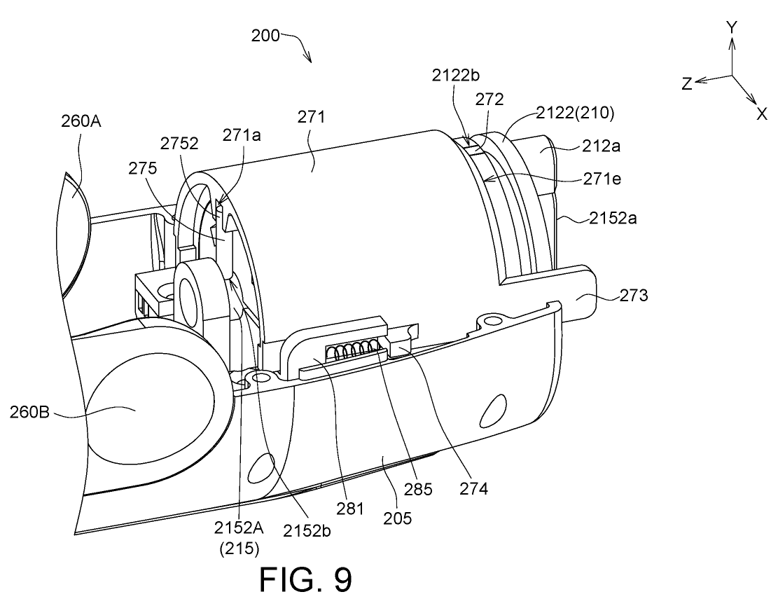

Referring to FIGS. 5A to 5B and 6 to 9, FIGS. 5A and 5B illustrate schematic diagrams of exploded views of the first steering module 200 in FIG. 1 along different viewing angles, FIG. 6 illustrates a schematic diagram of a cross-sectional view of the endoscope device 10 in FIG. 1 along a direction 6-6', FIG. 7 illustrates a schematic diagram of a bottom view of the endoscope device 10 in FIG. 1, and FIG. 8 illustrates a schematic diagram of a cross-sectional view of the endoscope device 10 in FIG. 1 along a direction 8-8', and FIG. 9 illustrates a partial schematic diagram of the interior of the first steering module 200 in FIG. 1.

As illustrated in FIGS. 5A and 5B, the first steering module 200 includes a module shell 205, a module outer rotating component 210, a module inner rotating component 215, a flexible tube 220, and a plurality of control wires (for example, a first control wire 230A, a second control wire 230B, a third control wire 230C and a fourth control wire 230D), a plurality of pulleys (for example, a first pulley 240A, a second pulley 240B, a third pulley 240C and a fourth pulley 240D), a first fastening portion 250A, a second fastening portion 250B, a first pressing plate 260A, a second pressing plate 260B, a first elastic component 265A (as illustrated in FIG. 8), a second elastic component 265B (as illustrated in FIG. 8), a locking component 270, a position-limiting rod 275, a base 280 and at least one third elastic component 285 (as illustrated in FIG. 9).

As illustrated in FIGS. 5A, 5B and 6, the module outer rotating component 210 includes a first module matching portion 212a. The module inner rotating component 215 passes through the module inner rotating component 215, is rotatable relative to the module inner rotating component 215 and includes a second module matching portion 2152a. Some of the control wires connect the flexible tube 220 with the module outer rotating component 210, and other of the control wires connect the flexible tube 220 with the module inner rotating component 215. As illustrated in FIG. 2A, the first module matching portion 212a and the second module matching portion 2152a are configured to connect with the base 100 of the endoscope device 10. For example, the first module matching portion 212a of the first steering module 200 may be connected with the first base matching portion 111a of the base 100, and the second module matching portion 2152a of the first steering module 200 may be connected with the second base matching portion 1151a (as illustrated in FIG. 2A) of the base 100.

In addition, the first module matching portion 212a of the first steering module 200 matches the first base matching portion 111a of the base 100 in shape, so that a gap between the first module matching portion 212a and the first base matching portion 111a is very small after the first module matching portion 212a and the first base matching portion 111a are connected with each other. Similarly, the second module matching portion 2152a of the first steering module 200 matches the second base matching portion 1151a of the base 100 in shape, so that a gap between the second module matching portion 2152a and the second base matching portion 1151a is very small after the second module matching portion 2152a and the second base matching portion 1151a are connected with each other. As a result, when the base 100 drives the base outer rotating component 110 and/or the base inner rotating component 115 to rotate, an impact noise between the base matching portion and the module matching portion may be reduced. In an embodiment, the maximum gap between the base matching portion and the module matching portion may be less than 0.1 millimeter (mm), for example, but may also be greater or less.

As illustrated in FIG. 5A, the module outer rotating component 210 includes an outer rotating wheel 211 and an outer shaft 212. The outer shaft 212 is fixed to the outer rotating wheel 211 and has an end surface 212e. The first module matching portion 212a is disposed on the end surface 212e of the outer shaft 212. The first module matching portion 212a is, for example, a protruding portion protruding relative to the end surface 212e. In the present embodiment, the number of the first module matching portions 212a is multiple, for example, two. The outer rotating wheel 211 has a shaft hole 211b1, a through hole 211b2 and a slot 211c, wherein the slot 211c connects the shaft hole 211b1 and a peripheral surface of the outer rotating wheel 211, and the through hole 211b2 extends to the slot 211c relative to the peripheral surface of the outer rotating wheel 211. Through the design of the slot 211c, the outer rotating wheel 211 is flexible (the through hole 211b2 may be slightly expanded or shrunk), which facilitates the outer shaft 212 to enter the shaft hole 211b1 effortlessly. The outer shaft 212 includes a first shaft portion 2121 and a second shaft portion 2122 connected with the first shaft portion 2121, wherein the first shaft portion 2121 is at least partially disposed in the shaft hole 211b1, and the second shaft portion 2122 has the aforementioned end surface 212e. After the first shaft portion 2121 of the outer shaft 212 enters the shaft hole 211b1 of the outer rotating wheel 211, a fixing component (for example, a screw) may pass through the through hole 211b2 and be fixed (for example, screwed) to a fixing hole (for example, screw hole) of the outer shaft 212 to fix the relative position of the outer shaft 212 and the outer rotating wheel 211. The aforementioned fixing component may shrink the slot 211c (or even the size of the slot 211c becomes 0), so that the first shaft portion 2121 of the outer shaft 212 is pressed into the shaft hole 211b1.

As illustrated in FIG. 5A, the module inner rotating component 215 includes an inner rotating wheel 2151 and an inner shaft 2152. The inner shaft 2152 is fixed to the inner rotating wheel 2151 and has an end surface 2152e. The second module matching portion 2152a is disposed on the end surface 2152e of the inner shaft 2152. The second module matching portion 2152a is, for example, a protruding portion protruding relative to the end surface 2152e. The inner rotating wheel 2151 has a shaft hole 2151b1, a through hole 2151b2 and a slot 2151c, wherein the slot 2151c connects the shaft hole 2151b1 with a peripheral surface of the inner rotating wheel 2151, and the through hole 2151b2 extends to the slot 2151c relative to the peripheral surface of the inner rotating wheel 2151. Through the design of the groove 2151c, the inner rotating wheel 2151 is flexible (the shaft hole 2151b1 may be slightly expanded or shrunk) to facilitate the inner shaft 2152 to enter the shaft hole 2151b1 effortlessly. The inner shaft 2152 includes a first shaft portion 2152A and a second shaft portion 2152B connected with first shaft portion 2152A, wherein the first shaft portion 2152A is at least partially disposed in the shaft hole 2151b1, and the second shaft portion 2152B has the aforementioned end surface 2152e. After the first shaft portion 2152A of the inner shaft 2152 enters the shaft hole 2151b1 of the inner rotating wheel 2151, a fixing component (for example, a screw) may pass through the through hole 2151b2 and be fixed (for example, screwed) to a fixing holes (for example, screw hole) of the inner shaft 2152 to fix the relative position of the inner shaft 2152 and the inner rotating wheel 2151. The aforementioned fixing member may reduce the slot 2151c (or even the size of the slot 2151c become 0), so that the first shaft portion 2152A of the inner shaft 2152 is pressed into the shaft hole 2151b1.

As illustrated in FIG. 5A and 6, the inner shaft 2152 may pass through the shaft hole 212b1 of the outer shaft 212 and the shaft hole 211b1 of the outer rotating wheel 211, and the module outer rotating component 210 may be disposed between the second shaft portion 2152B of the inner shaft 2152 and the inner rotating wheel 2151.

As illustrated in FIGS. 5A, 5B and 6, when the first steering module 200 is in a standby state (for example, the first steering module 200 has not yet been coupled to the base 100), the first module matching portion 212a and the second module matching portion 2152a may be arranged in a straight line. As a result, the first module matching portion 212a and the second module matching portion 2152a may match the second base matching portion 1151a and the first base matching portions 111a (as illustrated in FIG. 2A) which are also arranged in a straight line. Due to the design of the base matching portion and the module matching portion, the steering module may be quickly replaced.

As illustrated in FIG. 5A, the inner shaft 2152 further includes a first module positioning portion 2152C which may be disposed on the end surface 2152e of the inner shaft 2152 and protrudes relative to the end surface 2152e. In the present embodiment, the first module positioning portion 2152C may be connected to the second module matching portion 2152a. As illustrated in FIG. 2A, when the base 110 is assembled with the first steering module 200, the first module positioning portion 2152C of the first steering module 200 is connected to the first positioning portion 1151b1 of the base 110, so that the first module positioning portion 2152C triggers the first micro switch 130.

In the present embodiment, in the first steering module 200, the first control wire 230A, the second control wire 230B, the third control wire 230C and the fourth control wire 230D may be guided by the pulleys (the first pulley 240A, the second pulley 240B, the third pulley 240C and the fourth pulley 240D), and the turning of the flexible tube 220 may be controlled by the module outer rotating component 210 and/or the module inner rotating component 215. The further examples are given below.

As illustrated in FIGS. 5A, 5B and 6, the first control wire 230A and the second control wire 230B may be connected to the module outer rotating component 210. For example, the first pulley 240A and the second pulley 240B may be pivotally connected to a carrying seat 2051 of the module shell 205, wherein the outer rotating wheel 211 and the inner rotating wheel 2151 may be disposed on the carrying seat 2051. The first pulley 240A and the second pulley 240B may be respectively located on two opposite sides of the module inner rotating wheel 2151 of the module outer rotating component 210 (when the outer rotating wheel 211 and the inner rotating wheel 2151 are disposed on the carrying seat 2051). The first control wire 230A may extend to the module inner rotating component 215 through (e.g., winding) the first pulley 240A, and be fixed to the module inner rotating wheel 2151. As illustrated in FIG. 5B, for example, the module inner rotating wheel 2151 of the module inner rotating component 215 has a position-limiting hole 2151d1, and an end (not illustrated) of the first control wire 230A may pass through the position-limiting hole 2151d1 after winding around the inner rotating wheel 2151, and is fixed to a first end surface 2151e1 of the inner rotating wheel 2151 through a fixing portion (for example, glue). As illustrated in FIG. 5A, similarly, the module inner rotating wheel 2151 of the module inner rotating component 215 has a position-limiting hole 2151d2, and an end (not illustrated) of the second control wire 230B may pass through the position-limiting hole 2151d2 after winding around the position-limiting hole 2151d2 and is fixed to the second end surface 2151e2 of the outer rotating wheel 211 through a fixing portion (for example, glue). As a result, when the inner rotating wheel 2151 rotates, the length of the first control wire 230A and/or the second control wire 230B from the pulley to the flexible tube 220 may be controlled, thereby controlling the steering of the flexible tube 220. In the present embodiment, the first control wire 230A and the second control wire 230B may be connected to two opposite sides of the flexible tube 220 respectively, such as two opposite sides along one of Y-axis and X-axis. When the first control wire 230A and the second control wire 230B are connected to two opposite sides of the flexible tube 220 respectively along X-axis direction, a bending angle of the flexible tube 220 around Y-axis may be controlled. When the first control wire 230A and the second control wire 230B are connected to two opposite sides of the flexible tube 220 respectively along Y-axis, the bending angle of the flexible tube 220 around X-axis may be controlled.

As illustrated in FIGS. 5A, 5B and 6, the third control wire 230C and the fourth control wire 230D may be connected to the module outer rotating component 210. For example, the third pulley 240C and the fourth pulley 240D may be pivotally connected to the carrying seat 2051 of the module shell 205 and respectively disposed on two opposite sides of the rotating wheel 211 of the module outer rotating component 210. The third control wire 230C may extend to the module outer rotating component 210 through (e.g., winding) the third pulley 240C, and be fixed to the module outer rotating component 210. As illustrated in FIG. 5B, for example, the outer rotating wheel 211 of the module outer rotating component 210 has a position-limiting hole 211d1, and an end (not illustrated) of the third control wire 230C may pass through the position-limiting hole 211d1 after winding around the outer rotating wheel 211, and is fixed to the first end surface 211e1 of the outer rotating wheel 211 through a fixing portion (for example, glue). As illustrated in FIG. 5A, similarly, the outer rotating wheel 211 of the module outer rotating component 210 has a position-limiting hole 211d2, and an end (not illustrated) of the fourth control wire 230D may pass through the position-limiting hole 211d2 after winding around the outer rotating wheel 211, and is fixed to a second end surface 211e2 of the outer rotating wheel 211 through a fixing portion (for example, glue). As a result, when the outer rotating wheel 211 rotates, a length of the third control wire 230C and/or the fourth control wire 230D from the pulley to the flexible tube 220 may be controlled, thereby controlling the steering of the flexible tube 220. In the present embodiment, the third control wire 230C and the fourth control wire 230D may be connected to two opposite sides of the flexible tube 220 respectively, such as two opposite sides along the other one of Y-axis and X-axis. When the third control wire 230C and the fourth control wire 230D are connected to two opposite sides of the flexible tube 220 respectively along X-axis, the bending angle of the flexible tube 220 around Y-axis may be controlled. When the third control wire 230C and the fourth control wire 230D are connected to two opposite sides of the flexible tube 220 respectively along Y-axis, the bending angle of the flexible tube 220 around X-axis may be controlled.

As illustrated in FIGS. 4, 5A, 5B and 7, the module shell 205 accommodates the module outer rotating component 210, the module inner rotating component 215, and each control wire (for example, the first control wire 230A, the second control wire 230B, the third control wire 230C and the fourth control wire 230D). As illustrated in FIG. 7, the module shell 205 has a slide groove 205r, a first through hole 205a1 and a second through hole 205a2. The first through hole 205a1 and the second through hole 205a2 inwardly penetrate the module shell 205 from a bottom surface 205r1 of the slide groove 205r. The first fastening portion 250A protrudes from the first through hole 205a1, and the second fastening portion 250B protrudes from the second through hole 205a2. As a result, when the first steering module 200 is assembled with the base 100, the first fastening portion 250A and the second fastening portion 250B of the first steering module 200 may be respectively engaged with the first fastening groove 145r1 and the second fastening groove 145r2 of the base 100 (the first fastening groove 145r1 and the second fastening groove 145r2 are illustrated in FIG. 4) to fix the relative position between the base 100 and the first steering module 200, for example, to constrain the displacement freedom of the base 100 and the first steering module 200 along XZ plane.

As illustrated in FIGS. 7 and 4, the first through hole 205a1 and/or the second through hole 205a2 are elongated holes, for example. A long axis direction of the first through hole 205a1 (for example, the direction intersecting with Z-axis) may intersect with an extension direction (for example, Z-axis) of the slide groove 205r, and/or a long axis direction of the second through hole 205a2 (For example, the direction intersecting with Z-axis) may intersect with the extension direction of the slide groove 205r (e.g., Z-axis). The first through hole 205a1 and the second through hole 205a2 may be connected. For example, the first through hole 205a1 and the second through hole 205a2 may extend toward a central axis AX of the slide groove 205r and be connected to the central axis AX of the slide groove 205r. In the present embodiment, the first through hole 205a1 and the second through hole 205a2 are respectively disposed on two opposite sides of the central axis AX of the slide groove 205r. In another embodiment, the first through hole 205a1 and the second through hole 205a2 may not be connected.

As illustrated in FIGS. 7 and 4, an extension path of the first through hole 205a1 may be substantially consistent with or substantially overlap a movement trajectory of the first fastening portion 250A. As a result, when the first fastening portion 250A slides along the first through hole 205a1, the first fastening portion 250A does not interfere excessively with a side wall of the first through hole 205a1. Similarly, an extension path of the second through hole 205a2 may be substantially consistent with or substantially overlap with a movement trajectory of the second fastening portion 250B. As a result, when the second fastening portion 250B slides along the second through hole 205a2, the second fastening portion 250B does not interfere excessively with a side wall of the second through hole 205a2.

The first fastening portion 250A and the second fastening portion 250B may be connected to the module shell 205. As illustrated in FIGS. 5A and 5B, for example, the first pressing plate 260A connects the first fastening portion 250A with the module shell 205, and a first free end 260A1 of the first pressing plate 260A is movable relative to a connection between the first pressing plate 260A and the module shell 205. The first fastening portion 250A may be disposed at the first free end 260A1. As a result, when the first pressing plate 260A bears a force, the first pressing plate 260A may drive the first free end 260A1 to move, and then drive the first fastening portion 250A located on the first free end 260A1 to move. As illustrated in FIGS. 5A and 5B, similarly, the second pressing plate 260B connects the second fastening portion 250B and the module shell 205, and a second free end 260B1 of the second pressing plate 260B is movable relative to a connection between the second pressing plate 260B and the module shell 205. The second fastening portion 250B may be disposed at the second free end 260B1. As a result, when the second pressing plate 260B bears a force, the second pressing plate 260B may drive the second free end 260B1 to move, and then drive the second fastening portion 250B located on the second free end 260B1 to move.

As illustrated in FIG. 5A, the module shell 205 includes a lower shell 205C. The first pressing plate 260A and the second pressing plate 260B have a first connection portion 260A2 and a second connection portion 260B2 respectively. The first connection portion 260A2 and the second connection portion 260B2 may be pivotally connected to a connection portion 205C1 and 205C2 of the lower shell 205C respectively. The connection portion here is, for example, a pivot portion or a fixed portion. In an embodiment, the first connection portion 260A2 and/or the second connection portion 260B2 is, for example, one of the pivot shaft and the shaft hole, and the connecting portion 205C1 and/or 205C2 is, for example, the other one of the pivot shaft and the shaft hole.

As illustrated in FIGS. 5A, 5B and 7, the first pressing plate 260A and the second pressing plate 260B are disposed on two opposite sides of the slide groove 205r respectively. As a result, when the first pressing plate 260A and the second pressing plate 260B bear two forces approaching each other, the first free end 260A1 and the second free end 260B1 approach the slide groove 205r to drive the first fastening portion 250A and the second fastening portion 250B approaches toward the direction of the central axis AX of the slide groove 205r until the first fastening portion 250A and the second fastening portion 250B overlap the central axis AX of the slide groove 205r. When the first fastening portion 250A and the second fastening portion 250B overlap the central axis AX of the slide groove 205r, the overlapping first fastening portion 250A and second fastening portion 250B may slide in the guide groove 145g (the guide groove 145g is illustrated in FIG. 2A) of the base 100 until the first fastening portion 250A is aligned with the first fastening groove 145r1, and the second fastening portion 250B is aligned with the second fastening groove 145r2. At this time, the force exerted on the first fastening portion 250A and the second fastening portion 250B may be released, so that the first free end 260A1 and the second free end 260B1 are reset, so as to drive the first fastening portion 250A and the second fastening portion 250B enter the first fastening groove 145r1 and the second fastening groove 145r2 respectively. At this point, the fastening between the fastening of the base 100 and the first steering module 200 is completed.

As illustrated in FIGS. 5A, 5B and 8, the module shell 205 includes a first shell portion 205A and a second shell portion 205B. The aforementioned first pressing plate 260A and the second pressing plate 260B are connected to the first shell portion 205A and a second shell portion 205B respectively. The first shell portion 250A and the second shell portion 205B are disposed on two opposite sides of the module outer rotating component 210 and the module inner rotating component 215 (like lateral shells). There is a first accommodation space R1 between the first pressing plate 260A and the first shell portion 250A, and the first elastic component 265A may be disposed in the first accommodation space R1. When the first pressing plate 260A moves relative to the first shell portion 250A, the first elastic component 265A deforms and stores an elastic potential energy. When the first pressing plate 260A is released, the first elastic component 265A releases the elastic potential energy and returns to its original position. Similarly, there is a second accommodation space R2 between the aforementioned second pressing plate 260B and the second shell portion 205B, and the second elastic component 265B may be disposed in the second accommodation space R2. When the second pressing plate 260B moves relative to the second shell portion 205B, the second elastic component 265B deforms and stores an elastic potential energy. When the second pressing plate 260B is released, the second elastic component 265B releases the elastic potential energy and returns to its original position.

As illustrated in FIGS. 5A and 5B, when the first steering module 200 is in the standby state, the relative positions of the module outer rotating component 210, the module inner rotating component 215 and the module shell 205 may be in a locked state. The locking component 270 is slidably connected to the module shell 205 and is configured to selectively lock or relieve the module outer rotating component 210 and the module inner rotating component 215. For example, the locking component 270 includes a body 271, a position-limiting portion 272, at least one pressing portion 273 and at least one position-limiting portion 274. The position-limiting portion 272 is disposed on an end surface 271e of the body 271 and protrudes relative to the end surface 271e. When the first steering module 200 is in the standby state, the position-limiting portion 272 is located in a position-limiting hole 2122b of the second shaft portion 2122 of the outer shaft 212 of the module outer rotating component 210 to restrict the relative rotational freedom of the module outer rotating component 210 and the locking component 270. The locking component 270 is slidably but non-rotatably disposed relative to the module shell 205. As a result, when the module outer rotating component 210 and the locking component 270 cannot rotate relative to each other, the module outer rotating component 210 and the module shell 205 also cannot rotate relative to each other.

As illustrated in FIGS. 5A, 5B and 9, the first shaft portion 2152A of the inner shaft 2152 of the module rotating component 215 has a fixing hole 2152b, and the position-limiting rod 275 has a first end 2751 (the first end 2751 is illustrated in FIG. 5A) and the second end 2752, the first end 2751 may be fixed to the fixing hole 2152b of the inner shaft 2152. The body 271 has a position-limiting hole 271a, the position of which corresponds to the position-limiting rod 275. When the first steering module 200 is in the standby state, the second end 2752 of the position-limiting rod 275 may enter the position-limiting hole 271a of the body 271 to restrict the relative rotational freedom of the module rotating component 215 and the locking component 270. Since the locking component 270 is disposed on the module shell 205 in a slidable but non-rotatable manner relative to the module shell 205, when the module rotating component 215 and the locking component 270 cannot rotate relative to each other, the module rotating component 215 and the module shell 205 also cannot rotate relative to each other.

As illustrated in FIG. 9, the pressing portion 273 of the body 271 is disposed on the end surface 271e of the body 271 and protrudes relative to the end surface 271e. When the first steering module 200 moves toward the base 100, the pressing portion 273 contacts the base 100, and the body 271 is forced to slide relative to the module shell 205 (for example, toward +Z axis) until the position-limiting hole 271a of the body 271 is separated from the second end 2752 of the position-limiting rod 275, and the position-limiting portion 272 of the body 271 is separated from the position-limiting hole 2122b of the module outer rotating component 210 to relieve (or unlock) the locked state among the module outer rotating component 210, the module inner rotating component 215 and the module shell 205.

As illustrated in FIGS. 9 and 2B, the pressing portion 273 may selectively protrude or retract relative to the end surface 205e of the module shell 205. For example, when the first steering module 200 and the base 100 are successfully assembled, the pressing portion 273 is retracted relative to the end surface 205e of the module shell 205. When the first steering module 200 is separated from the base 100, the pressing portion 273 protrudes relative to the end face 205e of the module shell 205. When the pressing portion 273 protrudes relative to the end surface 205e of the module shell 205, the relative positions among the module outer rotating component 210, the module inner rotating component 215 and the module shell 205 are in the locked state.

As illustrated in FIGS. 9 and 5A, the base 280 is fixed to the module shell 205. The aforementioned locking component 270 is slidably connected to the base 280 but is non-rotatably connected to the base 280. The base 280 includes at least one position-limiting portion 281, and a third elastic component 285 may be disposed between the position-limiting portion 281 and the position-limiting portion 274 of the body 271. When the locking component 270 slides relative to the base 285 toward +Z axis, the third elastic component 285 deforms and stores an elastic potential energy. When the locking component 270 is released, the third elastic component 285 releases the elastic potential energy and resets (e.g., standby state).

The structure of the second steering module 300 is described below.

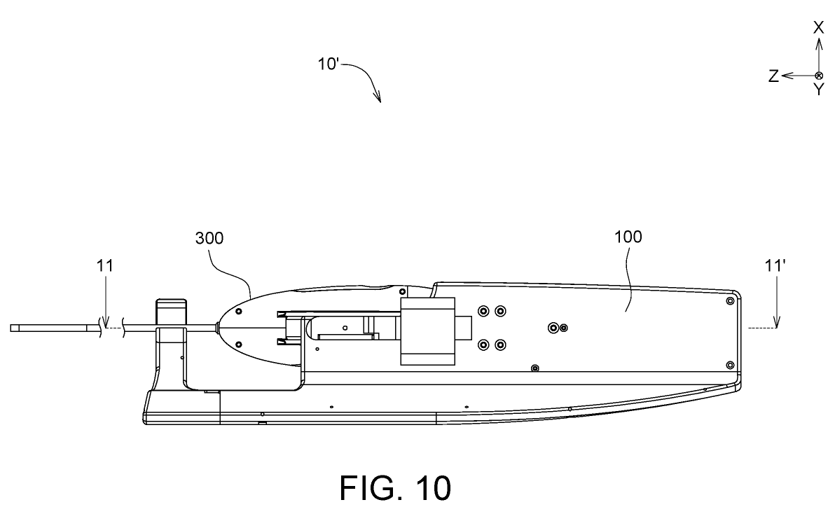

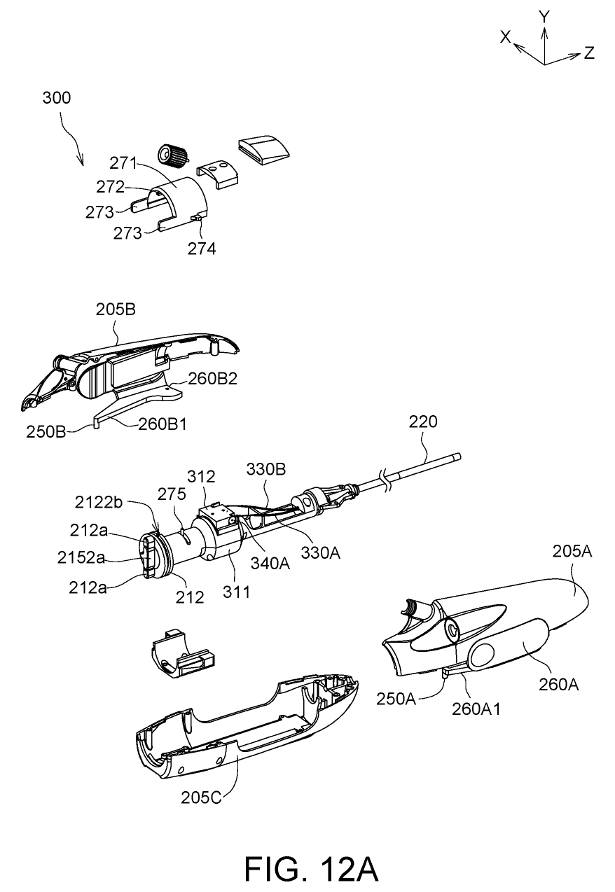

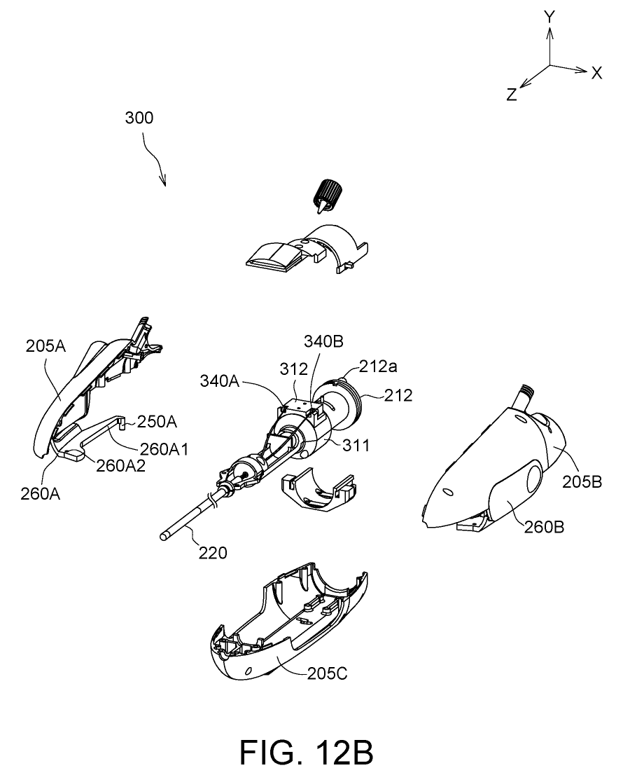

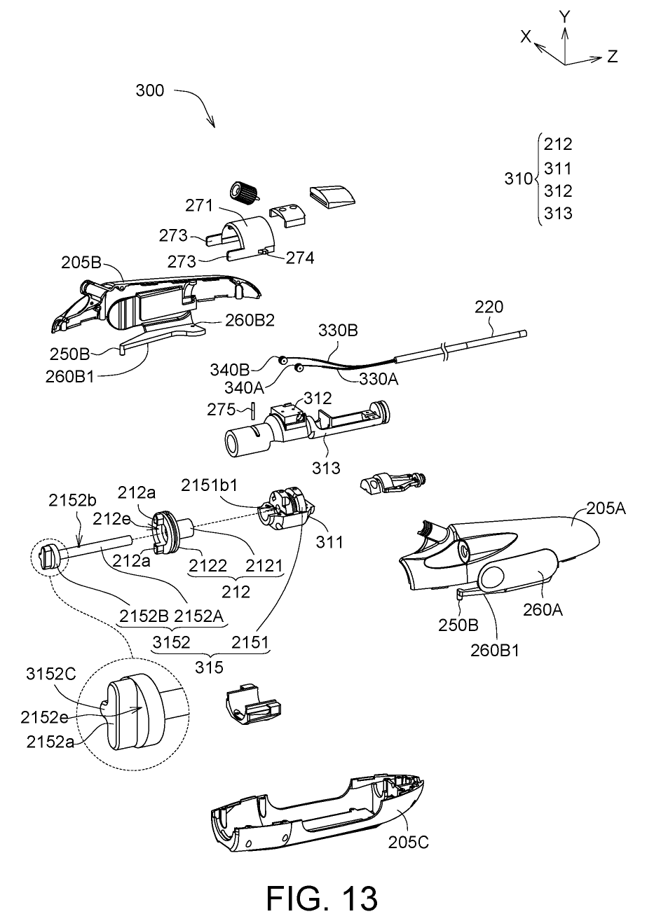

Referring to FIGS. 10, 11, 12A to 12B and 13, FIG. 10 illustrates a schematic diagram of a bottom view of the base 100 and the second steering module 300 in FIG. 1 after assembly, FIG. 11 illustrates a schematic diagram of a cross-sectional view of the endoscope device 10' along a direction 11-11', FIGS. 12A and 12B illustrate schematic diagrams of an exploded view of the second steering module 300 in FIG. 10 viewed from different viewing angles, and FIG. 13 illustrates a schematic diagram of an exploded view of the second steering module 300 in FIG. 12A.

As illustrated in FIGS. 12A to 12B, the second steering module 300 includes the module shell 205, the module outer rotating component 310, the module inner rotating component 315, the flexible tube 220, and a plurality of control wires (for example, a first control wire 330A and a second control wire 330B), a plurality of pulleys (for example, a first pulley 340A and a second pulley 340B), the first fastening portion 250A, the second fastening portion 250B, the first pressing plate 260A, the second pressing plates 260B, the first elastic component 265A (not illustrated), the second elastic component 265B (not illustrated), the locking component 270, the position-limiting rod 275, the base 280 (not illustrated) and at least one third elastic component 285 (not illustrated).

The second steering module 300 of the embodiment of the present disclosure includes the technical features the same as or similar to that of the aforementioned first steering module 200, and at least one difference is that the second steering module 300 and the first steering module 200 are different in the degree of freedom (DoF) of steering, and the module outer rotating component 310 of the second steering module 300 and the module outer rotating component 210 of the first steering module 200 are different in structure.

As illustrated in FIGS. 11, 12A to 12B, and 13, the module outer rotating component 310 includes the first module matching portion 212a. The module inner rotating component 315 passes through the module outer rotating component 310, is rotatable relative to the module outer rotating component 310 and includes the second module matching portion 2152a. These control wires (for example, the first control wire 330A and the second control wire 330B) connect the flexible tube 220 with the module rotating component 315. The first module matching portion 212a and the second module matching portion 2152a are configured to connect with the base 100 of the endoscope device 10'. For example, the first module matching portion 212a of the second steering module 300 may be connected with the first base matching portion 111a of the base 100, and the second module matching portion 2152a of the second steering module 300 may be connected with the second base matching portion 1151a of the base 100 (as illustrated in FIG. 2A).

As illustrated in FIGS. 11, 12A to 12B and 13, the first module matching portion 212a of the second steering module 300 matches the first base matching portion 111a of the base 100 in shape, so that the gap between the first module matching portion 212a and the first base matching portion 111a is very small after the first module matching portion 212a and the first base matching portion 111a are connected. Similarly, the second module matching portion 2152a of the second steering module 300 matches the second base matching portion 1151a of the base 100 in shape, so that the gap between the second module matching portion 2152a and the second base matching portion 1151a is very small (as illustrated in FIG. 2A) after the second module matching portion 2152a and the second base matching portion 1151a are connected. As a result, when the base 100 drives the base outer rotating component 110 and/or the base inner rotating component 115 to rotate, the impact noise of the base matching portion and the module matching portion may be reduced. In an embodiment, the maximum gap between the base matching portion and the module matching portion may be less than 0.1 mm, for example, but may also be greater or less.

As illustrated in FIG. 13, compared with the aforementioned module external rotating component 210, the module external rotating component 310 of the embodiment of the present disclosure may omit the external rotating wheel 211. For example, the module outer rotating component 310 includes the outer shaft 212, a rotating component bearing 311, a pulley seat 312 and at least one covering shell 313. The outer shaft 212 has the end surface 212e. The first module matching portion 212a is disposed on the end surface 212e of the outer shaft 212. The first module matching portion 212a is, for example, a protruding portion protruding relative to the end surface 212e. In the present embodiment, the number of the first module matching portions 212a is multiple, for example, two. The outer shaft 212 includes a first shaft portion 2121 and a second shaft portion 2122 connected with the first shaft portion 2121, wherein the first shaft portion 2121 may abut against the rotating component carrying seat 311, and the covering shell 313 may be assembled to the rotating component carrying seat 311 to cover the inner rotating wheel 2151 and the first shaft portion 2121. The second shaft portion 2122 has the aforementioned end surface 212e.

As illustrated in FIG. 13, the module inner rotating component 315 includes the inner rotating wheel 2151 and the inner shaft 3152. The inner shaft 3152 is fixed to the inner rotating wheel 2151 and has the end surface 2152e. The second module matching portion 2152a is disposed on the end surface 2152e of the inner shaft 3152. The second module matching portion 2152a is, for example, a protruding portion protruding relative to the end surface 2152e. The inner shaft 3152 may pass through the shaft hole 212b1 of the outer shaft 212 and be fixed to the shaft hole 2151b1 of the inner rotating wheel 2151. The module outer rotating component 310 may be disposed between the second shaft portion 2152B of the inner shaft 3152 and the inner rotating wheel 2151.

As illustrated in FIGS. 11, 12A to 12B and 13, when the second steering module 300 is in the standby state (for example, the second steering module 300 is not coupled to the base 100), the first module matching portions 212a and the second module matching portion 2152a may be arranged in a straight line. As a result, the first module matching portions 212a and the second module matching portion 2152a may match with the second base matching portion 1151a and the first base matching portions 111a (as illustrated in FIG. 2A) of the base 100 which are arranged in a straight line.

As illustrated in FIG. 13, the inner shaft 3152 further includes a second module positioning portion 3152C which may be disposed on the end surface 2152e of the inner shaft 3152 and protrudes relative to the end surface 2152e. In the present embodiment, the second module positioning portion 3152C may be connected to the second module matching portion 2152a. When the base 110 is assembled with the second steering module 300, the second module positioning portion 3152C of the second steering module 300 is connected to the second positioning portion 1151b2 of the base 110, so that the second module positioning portion 3152C triggers the second micro toggle switch 135 (as illustrated in FIG. 2A).

In the present embodiment, in the second steering module 300, the first control wire 330A and the second control wire 330B may be guided by the pulleys (for example, the first pulley 340A and the second pulley 340B) pivotally connected to the module outer rotating component 310, and the turning of the flexible tube 220 may be controlled by the module inner rotating component, as further illustrated below.

As illustrated in FIGS. 11, 12A to 12B and 13, the first control wire 330A and the second control wire 330B may be connected to the module rotating component 315. For example, the first pulley 340A and the second pulley 340B may be pivotally connected to the pulley base 312 and are located on two opposite sides of the pulley base 312 respectively. The first control wire 330A extends to the module inner rotating component 315 through (for example, winding around) the first pulley 340A and is fixed to the inner rotating wheel 2151 of the module inner rotating component 315. The way in which the first control wire 330A is fixed to the rotating wheel 2151 of the module inner rotating component 315 is the same as the way in which the first control wire 230A is fixed to the rotating wheel 2151 of the module inner rotating component 215, and it will not be repeated here. Similarly, the second control wire 330B extends to the module inner rotating component 315 through (e.g., winding around) the second pulley 340B and is fixed to the inner rotating wheel 2151 of the module inner rotating component 315. The way in which the second control wire 330B is fixed to the rotating wheel 2151 of the module inner rotating component 315 is the same as the way in which the first control wire 230B is fixed to the rotating wheel 2151 in the module inner rotating component.

When the module inner rotating component 315 rotates, a length of the first control wire 330A and/or the second control wire 330B from the pulley to the flexible tube 220 may be controlled, thereby controlling the steering of the flexible tube 220. The first control wire 330A and the second control wire 330B are connected to two opposite sides of the flexible tube 220 respectively. When the first control wire 330A and the second control wire 330B are connected to two opposite sides of the flexible tube 220 respectively along X-axis, the bending angle of the flexible tube 220 around Y-axis may be controlled. When the first control wire 330A and the second control wire 330B are connected to two opposite sides of the flexible tube 220 respectively along Y-axis, the bending angle of the flexible tube 220 around X-axis may be controlled. In addition, when the module inner rotating component 315 rotates synchronously with the module outer rotating component 310, the flexible tube 220 may rotate around the Z-axis without changing the bending angle.

In summary, the present disclosure proposes an endoscope device and a steering module thereof. The endoscope device includes a base. The base may be connected to a variety of different types of steering modules, such as a four-way steering module and a two-way steering module. In other words, a variety of the steering modules may share the same or one base. The base may be operated by hand, but may also be connected to a robotic arm, and the robotic arm may operate the endoscope device. The steering module may be a disposable module (for reducing the risk of secondary infection), so the endoscope device does not require disinfection procedures after each use, and the base may be reusable.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A steering module of an endoscope device, comprising:
a module outer rotating component comprising a first module matching portion;
a module inner rotating component passing through the module outer rotating component and being rotatable relative to the module outer rotating component, and comprising a second module matching portion;
wherein the first module matching portion and the second module matching portion are configured to connect with a base of the endoscope device.

2. The steering module according to claim 1, further comprising:
a flexible tube; and
a plurality of control wires, wherein some of the control wires connect the flexible tube with the module outer rotating component, and the others of the control wires connect the flexible tube with the module inner rotating component.

3. The steering module according to claim 1, further comprising:
a flexible tube; and
a plurality of control wires connecting the module inner rotating component with the flexible tube.

4. The steering module according to claim 1, wherein the module outer rotating component comprises:
an outer rotating wheel; and
an outer shaft fixed to the outer rotating wheel and having an end surface;
wherein the first module matching portion is disposed on the end surface of the outer shaft.

5. The steering module according to claim 1, wherein the module inner rotating component comprises:
an inner rotating wheel; and
an inner shaft fixed to the inner rotating wheel and having an end surface;
wherein the second module matching portion is disposed on the end surface of the inner shaft.

6. The steering module according to claim 1, wherein the module outer rotating component comprises:
an outer rotating wheel; and
an outer shaft fixed to the outer rotating wheel and having a shaft hole;
wherein the module inner rotating component comprises:
an inner rotating wheel; and
an inner shaft fixed to the inner rotating wheel and passing through the shaft hole of the outer shaft.

7. The steering module according to claim 1, further comprising:
a module shell accommodating the module outer rotating component, the module inner rotating component, the control wires, and having a slide groove and a first through hole, wherein the first through hole inwardly penetrates the module shell from a bottom surface of the slide groove;
a first fastening portion protruding from the first through hole.

8. The steering module according to claim 7, wherein the first through hole is an elongated hole.

9. The steering module according to claim 7, wherein the module shell has a second through hole, and the second through hole inwardly penetrates the module shell from the bottom surface of the slide groove; the steering module further comprises:
a second fastening portion protruding from the second through hole.

10. The steering module according to claim 9, wherein the second through hole is an elongated hole, and the first through hole and the second through hole extend to a central axis of the slide groove.

11. The steering module according to claim 9, further comprising:
a first pressing plate pivotally connected to the module shell and having a free end, wherein the free end is connected to the first fastening portion.

12. The steering module according to claim 1, further comprising:
a locking component slidably connected to the module shell and configured to selectively lock or release the module outer rotating component and the module inner rotating component.

13. The steering module according to claim 12, wherein the module shell has an end surface, and the locking component comprises a pressing portion selectively protruding or retracting relative to the end surface.

14. An endoscope device, comprising:
a steering module, comprising:
a module outer rotating component comprising a first module matching portion;
a module inner rotating component passing through the module outer rotating component and being rotatable relative to the module outer rotating component, and comprising a second module matching portion;
a base, comprising:
a base outer rotating component having a through hole and a first base matching portion; and
a base inner rotating component disposed in the through hole, being rotatable relative to the base outer rotating component and having a second base matching portion;
wherein the first base matching portion and the second base matching portion are configured to connect to the first module matching portion and the second module matching portion of the steering module respectively.

15. The endoscope device according to claim 14, wherein the steering module further comprises:
a flexible tube; and
a plurality of control wires, wherein some of the control wires connect the flexible tube with the module outer rotating component, and the others of the control wires connect the flexible tube with the module inner rotating component.

16. The endoscope device according to claim 14, wherein the steering module further comprises:
a flexible tube; and
a plurality of control wires connecting the module inner rotating component with the flexible tube.

17. The endoscope device according to claim 14, wherein the base further comprises:
a first driver connected to the base inner rotating component and configured to drive the base inner rotating component to rotate; and
a second driver connected to the base outer rotating component and configured to drive the configured to rotate.

18. The endoscope device according to claim 14, wherein one of the base inner rotating component and the base outer rotating component has a first positioning portion and a second positioning portion; the steering module is allowed to connect with one of the first positioning portion and the second positioning portion.

19. The endoscope device according to claim 14, wherein the base further comprising:
a base shell;
a slide rail disposed on the base shell and having a first fastening groove, wherein the fastening groove is configured to connect with the steering module.

20. The endoscope device according to claim 19, wherein the slide rail has a guide groove and a second fastening groove, and the second fastening groove is configured to connect with the steering module; the first fastening groove and the second fastening groove extend to the guide groove.

21. The endoscope device according to claim 14, wherein the base further comprises:
a first micro switch disposed on the base inner rotating component; and
a second micro switch disposed on the base inner rotating component;
wherein the steering module is allowed to be detected by one of the first micro switch and the second micro switch.
* * * * *
